(19) United States
(12) Patent Application Publication
(10) Pub. No.: US 2026/0151165 A1
(43) Pub. Date: Jun. 4, 2026

(54) Intramedullary Nail

(71) Applicant: Tyber Medical LLC, Bethlehem, PA (US)

(72) Inventors: Melissa Coale, Bethlehem, PA (US); Jeremy Rappenecker, Effort, PA (US); Logan Schleicher, Slatington, PA (US); William Fahey, Easton, PA (US); David Hannah, Nazareth, PA (US); Joseph Lazarowitz, Pottstown, PA (US); Ian Babcock, Bethlehem, PA (US); Victoria Bell, West Chester, PA (US); Matthew Gephart, Marquette, MI (US)

(73) Assignee: Tyber Medical LLC, Bethlehem, PA (US)

(21) Appl. No.: 19/407,009

(22) Filed: Dec. 3, 2025

Related U.S. Application Data
(60) Provisional application No. 63/727,734, filed on Dec. 4, 2024.

(51) Int. Cl. A61B 17/72; A61B 17/00

(52) U.S. Cl. CPC A61B 17/7233; A61B 17/725; A61B 2017/00867

(57) ABSTRACT

An intramedullary nail includes an elongate body having a proximal end and a distal body portion, and a longitudinal axis extending between the proximal end and the distal body portion. A securing member for locking the nail to a bone is provided, with the nail being inserted into the bone.

### BACKGROUND OF THE INVENTION

### Field of the Invention

The invention relates to a nail that is used to repair a fractured long bone.

### Description of the Related Art

Fractures of long bones, particularly in the lower extremities, can cause long lasting detrimental effects if not healed properly.

It would be beneficial to provide a nail that can be inserted into a fractured bone to connect the bone pieces and promote healing of the bone.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is an intramedullary nail comprising a body having a proximal end and a distal body portion, and a longitudinal axis extending between the proximal end and the distal body portion. A means for locking the nail to a bone is provided, with the nail being inserted into the bone.

In another embodiment, the present invention is an intramedullary nail comprising a body having a proximal end and a distal body portion, and a longitudinal axis extending between the proximal end and the distal body portion. A means for locking the nail to a bone is provided. The means extends between the proximal end and the distal end portion along the longitudinal axis.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective exploded view of an intramedullary nail according to an exemplary embodiment of the present invention;

FIG. 2 is a side elevational view, in section, of the tip of the nail of FIG. 1;

FIG. 3 is a top plan view of the main body of the nail of FIG. 1;

FIG. 4 is a side elevational view of the nail of FIG. 1 in a loaded condition;

FIG. 5 is a side elevational view of an intramedullary nail with staple according to an alternative embodiment of the present invention;

FIG. 6 is a front elevational view of the intramedullary nail of FIG. 5;

FIG. 7 is a side elevational view of an intramedullary nail with staple according to another alternative embodiment of the present invention;

FIG. 8 is a side elevational view of an intramedullary nail with staple according to a yet alternative embodiment of the present invention;

FIG. 9 is a side elevational view of an intramedullary nail with screw according to an alternative embodiment of the present invention;

FIG. 10 is a side elevational view of the nail and screw of FIG. 9, with the nail advanced over the screw;

FIG. 11 is a side elevational view of the nail and screw of FIG. 10, with a second screw added to secure the nail;

FIG. 12 is a top plan view of screwhead retaining cap according to an exemplary embodiment of the present invention;

FIG. 13 is a sectional view of the cap of FIG. 12 taken along lines 13-13 of FIG. 12;

FIG. 14 is a bottom plan view of the cap of FIG. 12;

FIG. 15 is a side elevational view of a nail body for use with the cap of FIG. 12;

FIG. 16 is a sectional view of the nail of FIG. 15, taken along lines 16-16 of FIG. 15;

FIG. 17 is a side elevational view of a screw used with the cap of FIG. 12 and the nail of FIG. 15;

FIG. 18 is a top plan view of the screw of FIG. 17;

FIG. 19 is a side elevational view of the cap of FIG. 12 being applied to the screw of FIG. 17;

FIG. 20 is a side elevational view, in section, of the cap and screw of FIG. 19, with the cap being pushed onto the head of the screw;

FIG. 21 is a side elevational view, in section of the cap of FIG. 19, fully inserted onto the screw of FIG. 17;

FIG. 22 is a top plan view of a screwhead retaining cap according to an alternative exemplary embodiment of the present invention;

FIG. 23 is a sectional view of the cap of FIG. 22, taken along lines 23-23 of FIG. 22;

FIG. 24 is a side elevational view of a nail body for use with the cap of FIG. 22;

FIG. 25 is a sectional view of the nail of FIG. 24, taken along lines 25-25 of FIG. 24;

FIG. 26 is a side elevational view of a screw used with the cap of FIG. 22 and the nail of FIG. 24;

FIG. 27 is an enlarged view of the head of the nail of FIG. 26 taken along circle 27 of FIG. 26;

FIG. 28 is a side elevational view of a screwhead retainer used with the screw of FIG. 26;

FIG. 29 is a sectional view of the retainer and screw of FIG. 28, taken along lines 29-29 of FIG. 28;

FIG. 30 is a side elevational view of the cap of FIG. 22 attached to the screw of FIG. 26;

FIG. 31 is a sectional view of the cap and crew of FIG. 30, taken along lines 31-31 of FIG. 30;

FIG. 32 is a side elevational view of a nail and head according to an alternative exemplary embodiment of the present invention;

FIG. 33 is a side elevational view of the nail and head of FIG. 32, with the head separated from the nail;

FIG. 34 is a side elevational view of the nail and head of FIG. 32, with the head drawn down to play out legs of the nail;

FIG. 35 is a side elevational view of another alternative embodiment of a nail and securing system implanted in a bone;

FIG. 36 is a side elevational view of still another alternative embodiment of a nail and securing system;

FIG. 37 is a side elevational view of another alternative embodiment of a nail with a securing system;

FIG. 38 is a side elevational view of yet another alternative embodiment of a nail and securing system;

FIG. 39 is a side elevational view of another alternative embodiment of a nail and securing system;

FIG. 40 is a side elevational view of an exemplary embodiment of a nail and securing system;

FIG. 41 is a side elevational view of another exemplary embodiment of a nail;

FIG. 42 is a side elevational view of another exemplary embodiment of a nail;

FIG. 43 is a side elevational view of another alternative exemplary embodiment of a nail and securing system;

FIG. 44 is a front elevational view of the nail and securing system of FIG. 43;

FIG. 45 is a front elevational view of the nail and securing system of FIG. 44 is a deployed position;

FIG. 46 is a side elevational view of an alternative embodiment of a nail;

FIG. 47 is a sectional view of the nail of FIG. 46;

FIG. 48 is a side elevational view of a nail according to an alternative embodiment;

FIG. 49 is a side elevational view of the nail of FIG. 48 in a deployed position;

FIG. 50 is a side elevational view of an alternative embodiment of a nail;

FIG. 51 is a side elevational view of the nail of FIG. 50 is a partially deployed position;

FIG. 52 is a side elevational view of the nail of FIG. 50 is a fully deployed position;

FIG. 53 is a side elevational view of an alternative embodiment of a nail in a bone;

FIG. 54 is a side elevational view of an alternative embodiment of a nail;

FIG. 55 is a side elevational view of the nail of FIG. 54 in a deployed position;

FIG. 56 is a side elevational view of another alternative embodiment of a nail;

FIG. 57 is a side elevational view of the nail of FIG. 56 in a deployed position;

FIG. 58 is a side elevational view of still another alternative embodiment of a nail;

FIG. 59 is a side elevational view of the nail of FIG. 58 in a deployed position;

FIG. 60 is a side elevational view of another alternative embodiment of a nail;

FIG. 61 is a front elevational view of the nail of FIG. 60;

FIG. 62 is a side elevational view of yet another alternative exemplary embodiment of a nail;

FIG. 63 is a side elevational view of an alternative embodiment of a nail;

FIG. 64 is a side elevational view of the nail of FIG. 63 in a pre-deployed position;

FIG. 65 is a side elevational view of the nail of FIG. 64 in a deployed position;

FIG. 66 is a side elevational view of an alternative embodiment of a nail;

FIG. 67 is a side elevational view of the nail of FIG. 66 in a deployed position;

FIG. 68 is a side elevational view of an alternative embodiment of a screw;

FIG. 69 is a sectional view of the screw of FIG. 68 taken along lines 69-69 of FIG. 68;

FIG. 70 is a side elevational view of the screw of FIG. 68 inserted into a nail;

FIG. 71 is a perspective view of a nail according to an alternative exemplary embodiment; and

FIG. 72 is a sectional view of the nail of FIG. 71, taken along lines 72-72 of FIG. 71.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or.” That is, unless specified otherwise, or clear from context, “X employs A or B” is intended to mean any of the natural inclusive permutations. that is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

An intramedullary nail, also known as an intramedullary rod, is a metal rod that is inserted into the bone marrow canal to treat long bone fractures. The present invention provides several exemplary embodiments of medullary nails for repairing bone fractures, such as, for example, but not limited to, tibial fractures, fibular fractures, femoral fractures, humerus fractures, ulnar fractures, and radial fractures. For simplicity, the bone not which any of the exemplary embodiments described below is inserted is referred to simply as “the bone.”

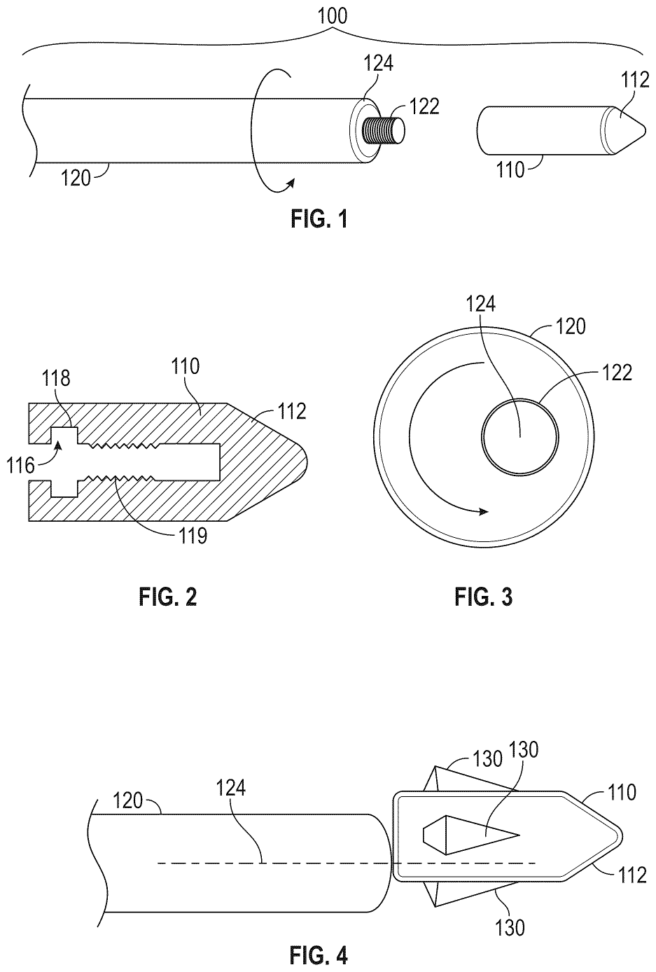

Referring now to FIGS. 1-4, an intramedullary nail 100 according to a first exemplary embodiment of the present invention is shown. Nail 100 is a two-part nail, with a proximal tip portion 110 and a distal body portion 120, with a longitudinal axis extending between the proximal end and the distal body portion.

Proximal tip portion 110 has a generally bullet shaped body with a proximal tip 112. A distal end 114 of tip portion 110 includes a passage 116 with an internal groove 118 and internal threads 119 between tip 112 and groove 118.

Body portion 120 includes a proximal threaded portion 122 sized to fit into passage 116 and engage internal threads 119. A lip 124 extends circumferentially round body portion 120 distal of threaded portion 122. Proximal tip portion 110 is threaded onto threaded portion 122 and, as proximal tip portion 110 is advanced toward body portion 120, internal groove 118 engages lip 124 so that proximal tip portion 110 cannot be threaded off body portion 120.

As shown in FIG. 3, threaded portion 122 is off-center of an axis 124 of body portion 120 so that, as tip portion 110 is rotated 180 degrees relative to body portion 120, tip 110 can eccentrically roll off-center, as shown in FIG. 4. Barbs 130 are radially located around an exterior of proximal tip portion 110. Barbs 130 dig into the interior of the bone into which nail 100 is being inserted. Distal body portion 120 can be rotated relative to proximal tip portion 110 to further dig barbs 130 into the bone. Barbs 130 can be formed to be permanently extending outwardly of proximal tip portion 110. Alternatively, barbs 130 can be constructed from a shape memory material so that, prior to insertion, barbs 130 are retracted into proximal tip portion 110. As barbs 130 heat up to body temperature after insertion, barbs 130 extend to the position shown in FIG. 4.

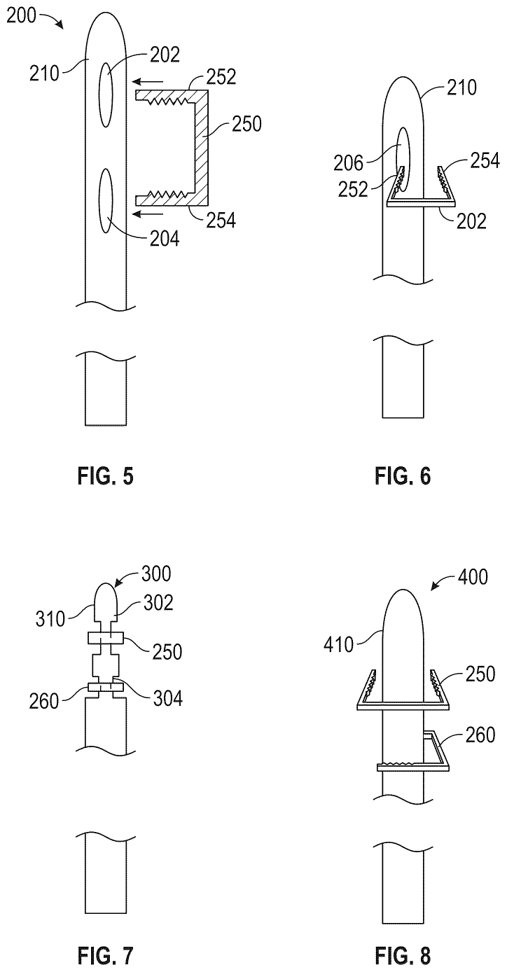

An alternative embodiment of an intramedullary nail 200 is shown in FIGS. 5 and 6. Nail 200 includes a plurality of through openings 202, 204, 206 in proximal end portion 210 of nail 200. Through openings 202, 204, 206 can be oblong in shape. Through openings 202, 204 can be longitudinally aligned and spaced so that a staple 250 can be inserted into through openings 202, 204 with a first leg 252 of staple 250 being inserted into through opening 202 and a second leg 254 of staple 250 inserted into through opening 204.

Optionally, through opening 206 can be provided. In an exemplary embodiment, through opening 206 is located and extends at a 90 degree angle relative to through openings 202, 204. A single leg 252 of staple 250 can be inserted into through opening 206, with remaining leg 254 of staple 2540 being inserted only into adjoining bone structure.

An alternative embodiment of a nail 300 according to the present invention is shown in FIG. 7. A proximal end portion 310 of nail 300 includes a plurality of flats or undercuts 302, 304 along the length of proximal end portion 310.

A first staple 250 can engage first flat 302 while a second staple 260 can engage a second flat 304. The legs of staples 250, 260 pass around proximal end portion 310 to engage bone structure (not shown). While two flats 302, 304 are shown, those skilled in the art will recognize that more or less than two flats can be provided. Further, while flats 302, 304 are shown to be formed on the same face of nail 300 with each other, one flat 304, 304 can be formed at a 90 degree or other angle relative to the other flat 304, 302.

Alternatively, instead of through openings 202, 204, 206 or flats 302, 304, a nail 400, shown in FIG. 8, can be provided without any special retention features. Staples 250, 260 are inserted around a proximal end portion 410 of nail 400. In an exemplary embodiment, staple 260 can be placed around nail 400 about 90 degrees around a side of nail 400 relative to staple 250.

For any of nails 200, 300, 400, the nail can be inserted into a bone (not shown) in a known manner with staples 250, 260 being inserted through the bone and into/around the nail to secure the nail to the bone. Fluoroscopy or other known visualization techniques can be used to locate the nail within the bone for proper placement of staples 250, 260.

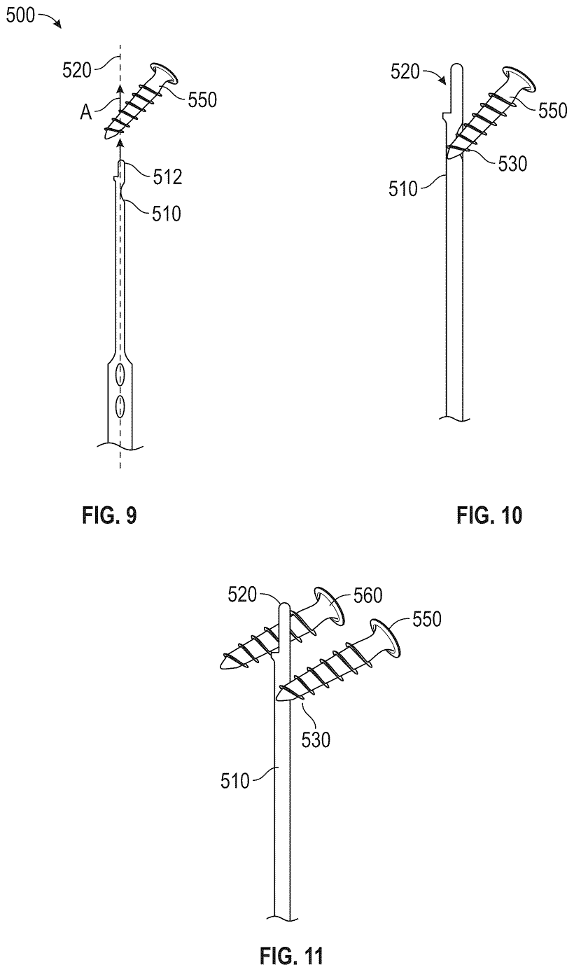

FIGS. 9-11 show a nail 500 according to another alternative embodiment of the present invention. Nail 500 includes a proximal end portion 510 having a flattened tip 520 relative to a remaining portion of proximal end portion 510. Flattened tip 520 allows for proximal end portion 510 to deflect while being inserted into the bone (not shown). A tangent cut 530 is provided distal of tip 520.

To insert nail 500 into a bone (not shown), a screw 550 is first inserted into the bone at an oblique angle relative to a longitudinal axis 520 of nail 500. In an exemplary embodiment, screw 550 is located at the tip of the lateral malleolus of the bone, plus the length of nail 500, minus a predetermined distance, such as, for example, 5 mm, corresponding to the location of tangent cut 530 relative to the end of tip 520 of nail 500.

Nail 50 is then advanced along axis 520 in the direction “A” of FIG. 9. As tip 520 encounters screw 550, tip 520 deflects around the tip of screw 550. Nail 500 continues along direction A until the tip of screw 550 engages tangent cut 530, wherein the advance of nail 500 is stopped. Screw 550 can then be advanced into tangent cut 530 to secure nail 500. A second screw 560 is inserted at tip 520, contralateral and tangent to tip 530, to prevent axial rotation of nail 500 inside the bone.

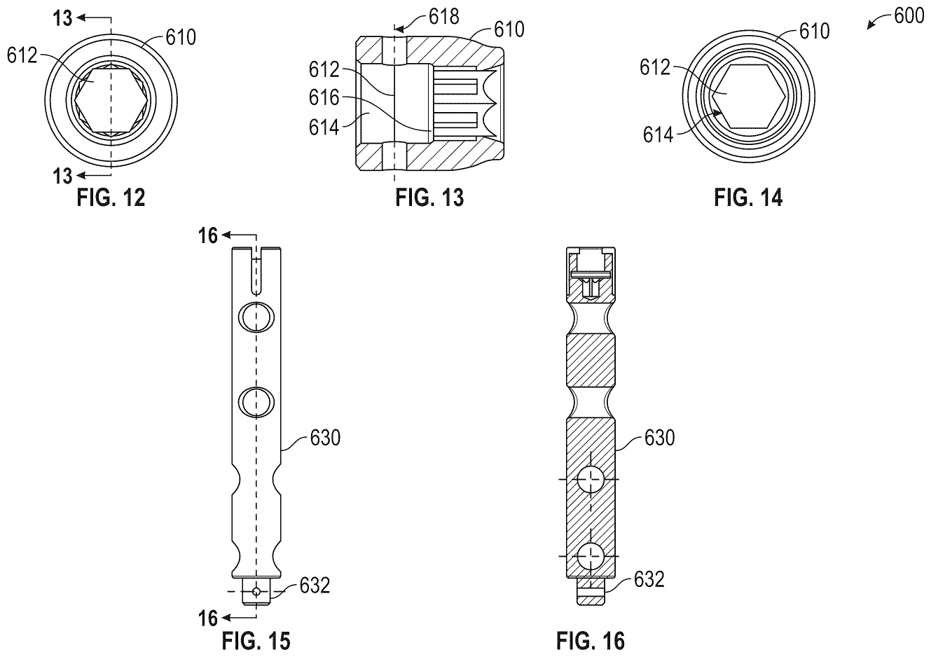

FIGS. 12-16 show an intramedullary screw and nail assembly 600 according to an exemplary embodiment of the present invention. Assembly 600 includes a cap 610 that fits over the head 632 of a nail 630. Cap 610 includes a central longitudinal through passage 612 with a bottom opening 614 sized to receive head 632 of nail 630. A shoulder 616 serves as a stop to prevent advancement of nail 630 further into cap 610. A transverse through passage 618 extends through the bottom portion 620 of head and cuts through passage 612. Transverse through passage 618 is sized to accept a pin (not shown).

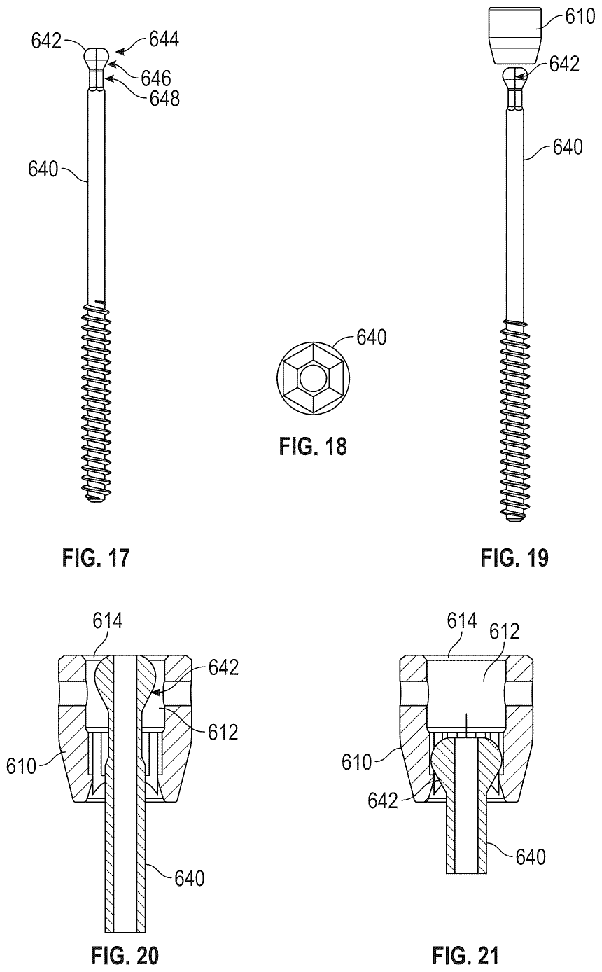

FIGS. 17-21 show a screw 640 and screw cap 610 used to secure screw 640. Screw 640 includes a head 642 having a convex upper surface 644 to allow screw 640 to articulate. Screw 640 can be cannulated. A lower surface 646 of head 642 is tapered to allow for articulation relative to cap 610. A tapered portion 648 below head 642 allows for articulation clearance when head 642 is inserted into cap 610.

FIGS. 20 and 21 show head 642 being lowered into through passage 612 through bottom opening 614. After screw 640 is inserted into cap 610, nail 630 can be inserted into cap 610 such that passage 632 in nail 630 aligns with passage 618 in cap. The use of cap 610 between screw 640 and nail 630 allows for relative articulation between screw 640 and nail 630 in a bone (not shown).

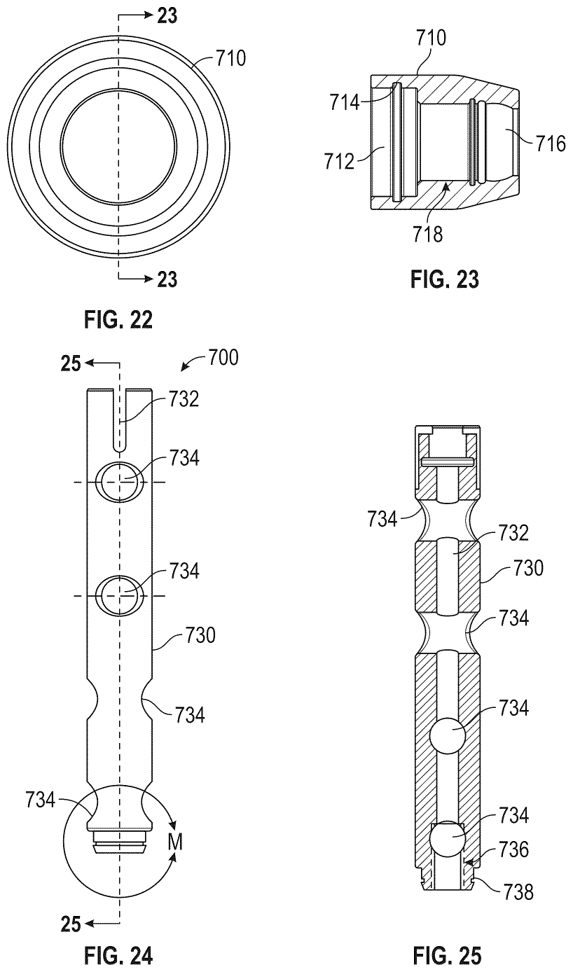

FIGS. 22-31 show an intramedullary screw and nail assembly 700 according to an alternative exemplary embodiment of the present invention. Assembly 700 includes a cap 710 that fits over the head 732 of a nail 730, shown in FIGS. 24 and 25. Cap 710 includes a central longitudinal through passage 712 with a bottom opening 714 sized to receive head 732 of nail 730. A bottom retaining groove 715 is cut in passage 712 proximate to bottom opening 714 to fit a nail retaining ring (not shown). Passage 712 also includes a top opening 716 sized to receive a screw 740. A top retaining groove 717 is cut in passage 712 proximate to top opening 716 to fit a screw retaining ring (not shown). A central passage 718 is threaded for a set screw 760 (shown in FIG. 31) to lock the head 742 of screw 740 in cap 710.

FIGS. 24 and 25 show nail 730. Nai 730 is cannulated with a through passage 732 and a plurality of cross passages 734, with two of passages 732 extending orthogonally relative to two other cross passages 734.

A bottom end of passage 732 includes a threaded region 736 that is sized to accept a set screw (not shown). Bottom end of nail 730 also includes a retaining ring groove 738 to receive the nail retaining ring.

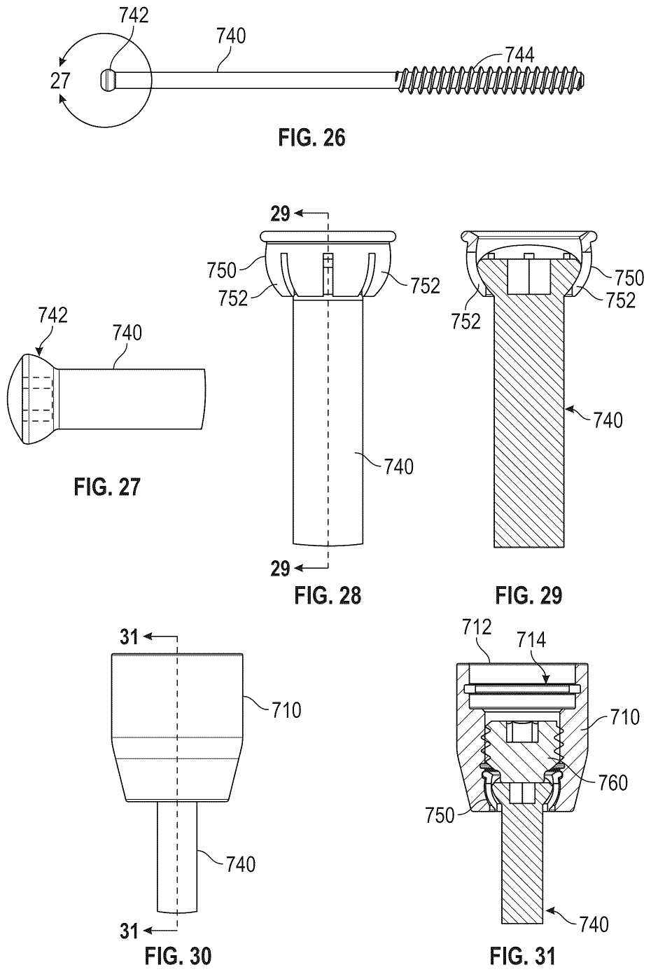

FIGS. 26 and 27 show the screw 740 used with assembly 700. Screw 740 includes a spherical head 742 and a threaded region 744 distal from head 742. A screw head retainer 750 is used with cap 710 and screw 740 and is shown in FIGS. 28-31.

Retainer 750 provides a spherical surface for articulation of screw 740 inside cap 710. Retainer 750 is slotted with individual fingers 752 to allow screw head 742 to seat inside retainer 750. FIG. 31 shows screw and 740 and retainer 750 inserted into cap 710. Although not shown, nail 730 is inserted into passage 712 at opening 714, forming the full nail assembly 700.

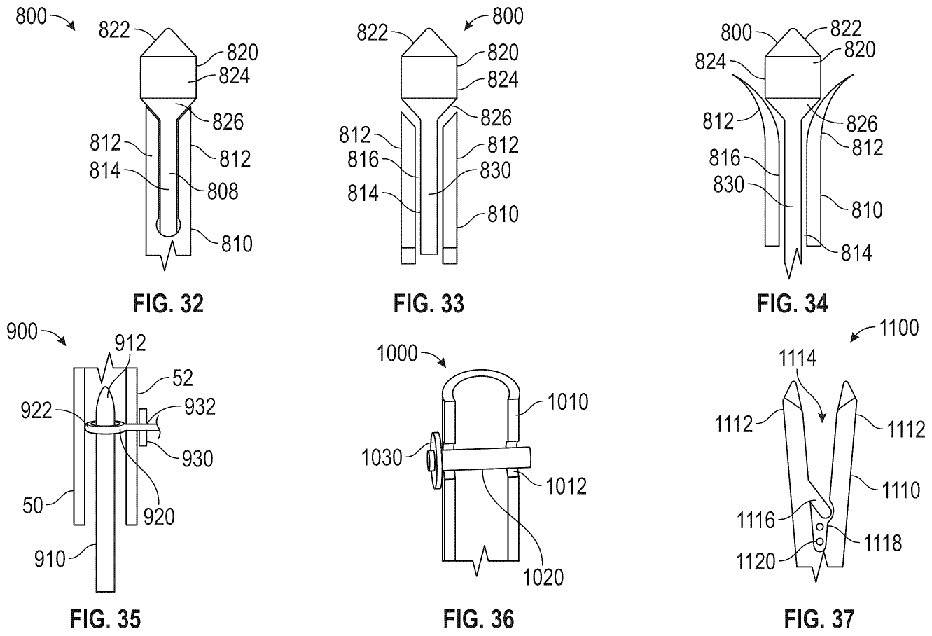

An alternative embodiment of a nail assembly 800 is shown in FIGS. 32-34. Nail assembly 800 includes a hollow nail 810 with a plurality of plurality of legs 812, with each pair of adjacent legs 812 split by a slot 814. Nail 810 also has a longitudinal cannula 816 formed therethrough in communication with slots 814.

A tapered insertion tip 820 is slidingly inserted into the end of nail 810. Tip 820 includes a conical free end 822 mounted on a cylindrical body 824. A conical tail 826 is attached to body 824, distal from tip 820. A rod 830 extends from tail 826 into cannula 816.

To secure nail 810 inside a bone, rod 830 is pulled downwardly through cannula 816. Tail 826 engages legs 812, and splays legs 812 outwardly such that nail 810 is forced against the inside cannula of the bone, retaining nail 810 inside the bone and preventing the nail 810 from rotating within the bone.

In an alternative embodiment, both rod 830 and legs 812 can have matching threads such that, rod 830 can be rotated to advance tip 820 downward into nail 810, thereby sharing the load used to splay legs 814 outwardly.

Another alternative embodiment of a nail assembly 900 is shown in FIG. 35. Nail assembly 900 includes a nail 910 that can be inserted into a bone 50. An opening 52 is formed in the bone 50 to expose the interior of bone 50. A suture 920 can be formed with a loop 922 to form a lasso through which the tip 912 of nail 910 can be inserted. A tension button 930 includes a through opening 92 through which suture 920 can extend. Button 930 can then be rotated to tighten lasso 922 around nail 910, preventing nail 910 from rotating inside bone 50.

Another alternative embodiment of a nail assembly 1000 is shown in FIG. 36. Nail assembly 1000 includes a nail 1010 having a transverse slot 1012 that extends across a diameter of nail 1010. A suture loop 1020 extends fully through slot 1012. A tightening button 1030 is located adjacent to slot 1012, with suture 1020 passing through button 1030. As button 1030 is rotated, suture 1020 is tightened, restricting nail 1010 from rotating inside of bone.

Still another alternative embodiment of a nail assembly 1100 is shown in FIG. 37. Assembly 1100 include a nail 1110 having at least two legs 1112 located on either side of a cannula 1114. A latch 1116 extends obliquely downwardly from one leg 1112 toward the other leg 1112. A small cut 1118 is formed in the other leg for latch 1116 to extend into.

A suture 1120 extends transversely through cannula 1114. Suture 1120 can be dual threads that loop back. Suture 1120 is inserted through the bone prior to insertion of the nail 1110. As the nail 1110 is advanced through the bone cavity, latch 1116 encounters suture 1120. Suture 1120 bends latch 1116 downward so that nail 1100 can continue being advanced. After latch 1116 fully passes suture 1120, latch 1116 returns to its original position, securing suture 1120 below latch 1116. Optionally, suture 1120 can be tightened by a button (not shown), similar to button 1030 discussed above.

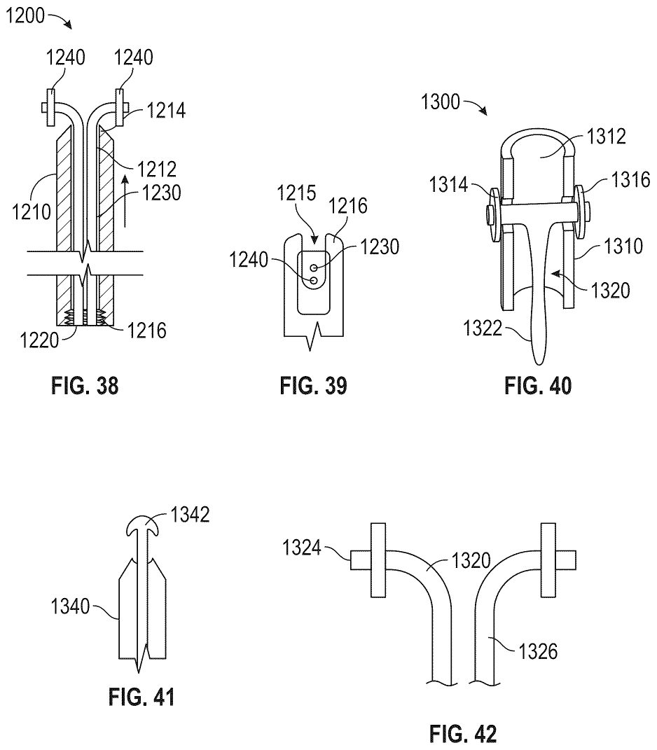

FIGS. 38 and 39 show another alternative embodiment of a nail assembly 1200. Assembly 1200 includes a nail 1210 having a through cannula 1212 with an open top end 1214 and a bottom end 1216 sealed by a set screw 1220. Top end 1214 includes a channel 1215 formed in nail 1210 on diametrically opposing sides of nail 1210.

A suture 1230 is attached to set screw 1220 and both ends of suture 1230 extend upwardly through cannula 1212 and out top end 1214, where the suture ends separate to opposite directions and through a respective channel 1215, where each end is secured to the bone (not shown) by buttons 1240.

FIGS. 40-42 show still another alternative embodiment of a nail assembly 1300. Assembly 1300 includes a nail 1310 having a through cannula 1312 formed therein. A looped suture 1320 is formed and attached to sides 1314, 1316 of nail 1310, with a lower portion 1322 of suture 1320 extending downwardly inside cannula 1312. Suture 1320 can be braided wire.

A puller 1340, shown in FIG. 41, can be inserted into cannula 1312 from the bottom to grasp lower portion 1322 of suture 1320. Puller 1340 includes a tee-shaped grabber 1342 that can be extended from puller 1340 to grasp lower portion 1322 of suture and retracted to pull suture 1320 downwardly to tighten suture 1320. FIG. 42 shows an alternative embodiment of a single suture with two free ends 1324, 1326, each secured by a button, such as button 1240 described above.

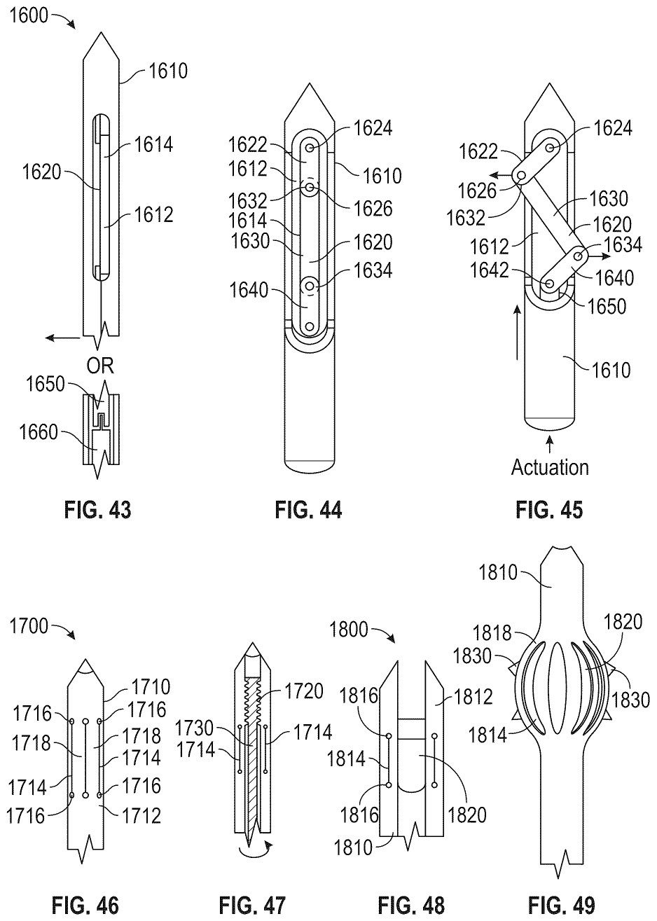

As shown in FIGS. 43-45, an alternative embodiment of a nail assembly 1600 is shown. Assembly 1600 includes a nail 1610 having a hollow body 1612 with diametrically opposed open slots 1614. A push rod assembly 1620 is provided inside body 1612. Push rod assembly 1620 includes a first link 1622 having a fixed end 1624 and a pivoting end 1626. Pivoting end 1626 is pivotally attached to a second, or central, link 1630 at a first end 1632. Pivoting link 1630 is attached to a third link 1640 at a second end 1634. Third link 1640 is attached to a pusher link 1650 at a pusher end 1642.

An actuator 1660 can extend upward into nail body 1612 to engage pusher link 1650 and advance pusher link 1650 toward first link 1620. Because fixed end 1624 of first link 1620 does not advance, links 1620, 1630, 1640 pivot with respect to each other such that pivoting end 1626 of first link 1620, second link 1630, and pusher end 1642 of third link 1640 all pivot through slots 1614 to an exterior of body 1612.

Pivoting end 1626 and pusher end 1642 engage the interior wall of the bone and engage the bone, preventing movement or rotation of nail assembly 1600 within the bone.

Referring now to FIGS. 46 and 47, an alternative exemplary embodiment of a nail assembly 1700 is provided. Assembly 1700 include a nail 1710 having a hollow body 1712 with a plurality of longitudinal slots 1714 formed around a periphery of body 1712. Each end of each slot 1714 can include a stress-relief cutout 1716. A rib 1718 is defined between each set of adjacent slots 1714.

Hollow body 1712 includes an internally threaded section 1720 above slots 1714. A threaded draw rod 1730 extends upwardly through nail body 1720, past slots 1714, and into threaded section 1720. Draw rod 1730 is configured such that rotation of draw rod 1730 does not advance or retract rod 1730 within body 1712. As a result, rotation of draw rod 1730 and its engagement with internal threads 1720 draws threaded section 1720 downward, compressing body 1720, which ultimately results in nail body compressing at slots 1714, with ribs 1718 bending outwardly to engage the bone canal and fix nail assembly 1700 against longitudinal translation and rotation within the bone.

Referring now to FIGS. 48 and 49, an alternative exemplary embodiment of a nail assembly 1800 is provided. Assembly 1800 include a nail 1810 having a hollow body 1812 with a plurality of longitudinal slots 1814 formed around a periphery of body 1812. Each end of each slot 1814 can include a stress-relief cutout 1816. A rib 1818 is defined between each set of adjacent slots 1814.

A balloon 1820 is provided inside nail body 1812 at slots 1814. Balloon 1820 can be in fluid communication with an inflation medium, such as saline. When nail assembly 1800 is in the desired location, balloon 1820 is inflated. As a result, the inflated balloon 1820 forces ribs 1818 to bend outwardly to engage the bone canal and fix nail assembly 1800 against longitudinal translation and rotation within the bone.

Optionally, for either of nail assemblies 1700, 1800, barbs 1830, shown in FIG. 49, can be provided on the exterior of ribs 1818 to dig into the bone matter to further prevent advancement or rotation of nail assembly 1700, 1800 within the bone.

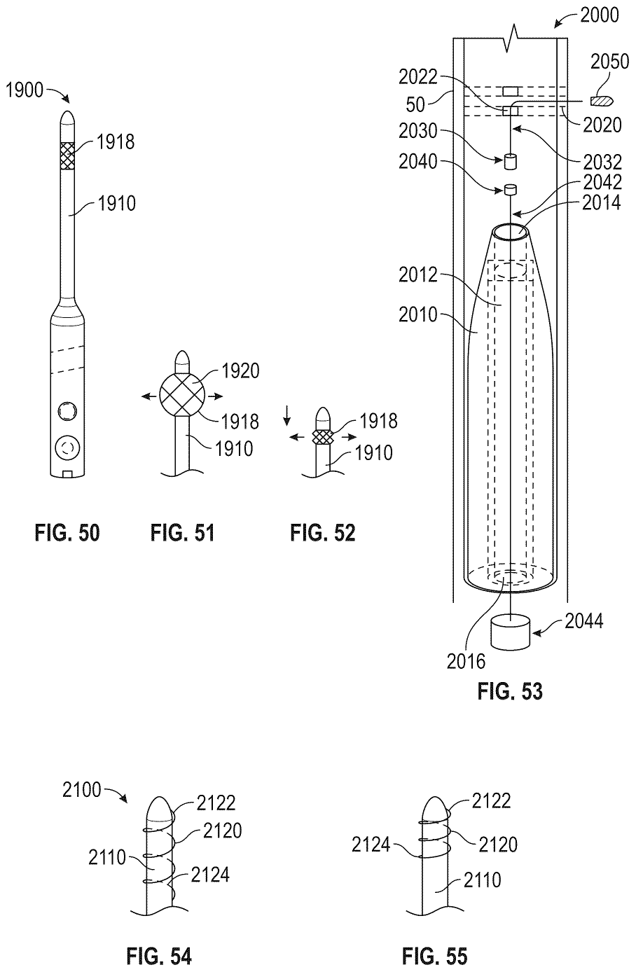

Referring now to FIGS. 50-52, an alternative exemplary embodiment of a nail assembly 1900 is shown. Assembly 1900 can be similar to assemblies 1700, 1800, but instead of ribs 1718 1818, a lattice structure 1918 is provided as part of nail body 1910. FIG. 51 shows a balloon 1920 expanding the lattice structure 1918, while FIG. 52 shows the same mechanism as for assembly 1700 to compress nail body 190 and force the lattice structure 1918 to expand outwardly.

FIG. 53 shows an alternative embodiment of a nail assembly 2000. Assembly 2000 uses a magnet to advance a suture that secures a nail 2010 inside bone 50. Nail 2010 includes a through cannula 2012. With a top opening 2014 and a bottom opening 2014.

Prior to inserting nail 2010 into bone 50, a cannulated screw 2020 is inserted into bone 50 above the final location of nail 2010. Screw 2020 has a transverse through opening 2022 sized to allow a magnet 2030 on a suture 2032 to pass through.

After screw 2020 is inserted, magnet 2030 is passed through opening 2022 and dropped down the length of bone 50. Magnet 2030 enters nail 2010 at top opening 2014 and passes out nail 2010 at bottom opening 2016. Magnet is attached to a second magnet 2040 attached to a second suture 2042. A plug 2044 is attached to the other end of suture 2042.

Nail 2010 is inserted into a desired location in bone 50. Suture 2032 is pulled from screw 2020 until plug 2044 engages bottom opening 2016 of nail 2010. At this point, both magnet 2030 and magnet 2040 are pulled from screw 2020, with only suture 2042 extending through nail 2010 and screw 2020. A threaded screw plug 2050 is threaded into screw 2020 to secure suture 2042, which, with plug 2044, prevents nail 2010 from moving or rotating within bone 50.

Another alternative embodiment of a nail assembly 2100 is shown in FIGS. 54 and 55. Assembly 2100 includes a nail 2110 with a shape memory metal coil 2120 coiled around nail 2110. The shape memory material can be Nitinol or other suitable material.

A top end 2122 of coil 2120 is fixedly attached to nail 2110 while a bottom end of coil 2120 is free. Prior to insertion of assembly 2100 into a bone, the coil 2120 can look like the coil in FIG. 54. After insertion, upon heating to body temperature, coil 2120 spring outward engaging the interior of the bone. The attached top end 2122 keeps the coil 2120 attached to nail 2010 while the free end 2124 moves, allowing coil 2120 to expand and engage the bone to secure assembly 2100 in place.

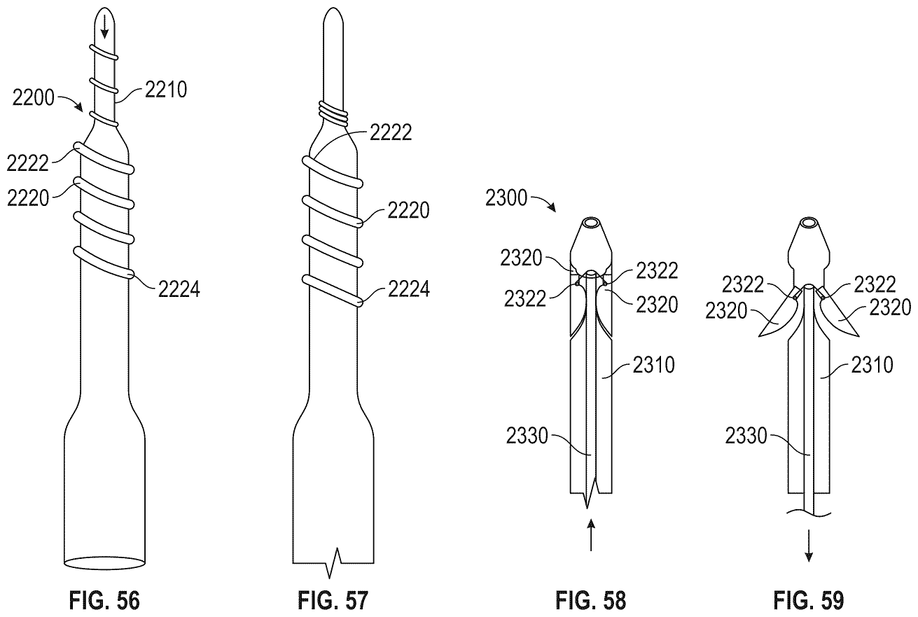

Another alternative embodiment of a nail assembly 2200 is shown in FIGS. 56 and 57. Similar to assembly 2100, assembly 2200 includes a nail 2210 with a shape memory metal coil 2220 coiled around nail 2210. The shape memory material can be a shape memory material, such as Nitinol or other suitable material.

A top end 2222 of coil 2220 is fixedly attached to nail 2210 while a bottom end of coil 2220 is free. Coil 2220 has a lattice structure so that, upon heating, structure expands from the condition shown in FIG. 56 to the condition in FIG. 57, wherein coil 2220 engages the interior of the bone to secure nail assembly 2200 to the bone.

Another alternative embodiment of a nail assembly 2300 is shown in FIGS. 58 and 59. Nail assembly 2300 includes a nail 2310 with two wings 2320 folded into body 2300 Wing 2310 are pivotally fixed on body 2310 at pivots 2322.

A push rod 2330 extends though nail body 2310 and is attached to pivots 2322. As push rod 2330 is drawn downwardly push rod 2330 pulls wings 2320 at pivots 2322, pivoting wings 2320 outward from the position shown in FIG. 58 to the position shown in FIG. 59, where the wings 2320 dig into the interior of the bone to secure the nail assembly 2300 in place inside the bone against rotation.

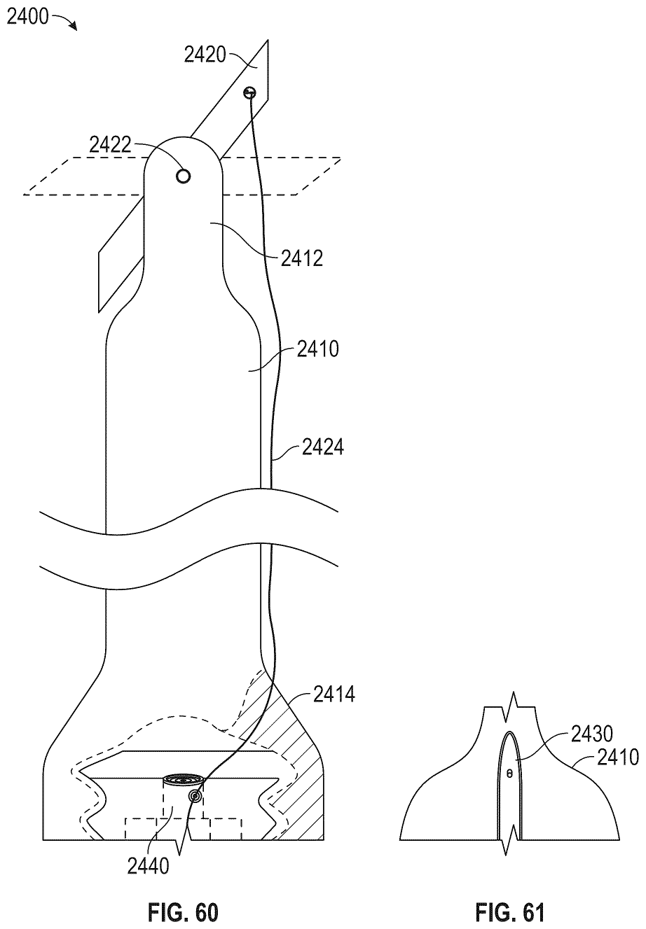

Referring now to FIGS. 60 and 61, a nail assembly 2400 according to another exemplary embodiment is shown. Nail assembly 2400 includes a nail 2410 having a top end 2412 and a bottom end 2414. A securing rod 2420 is rotationally attached to an axle 2422 at the top end 2412. A pull cable 2424 is attached to one end of rod 2420 and extends the length of nail 2410 to a slot 2430 at the bottom end 2414 of nail 2410.

Cable 2424 is attached to a set screw 2440 that is threaded int bottom end 2414. Set screw 2440 can have a reverse thread to pull cable 2424 as set screw 2440 is tightened. As cable 2424 is pulled, cable 2424 pulls on securing rod 2420, rotating securing rod 2420 to the position shown in dashed lines in FIG. 60, driving the opposing tips of securing rod 2420 into the side wall of the bone.

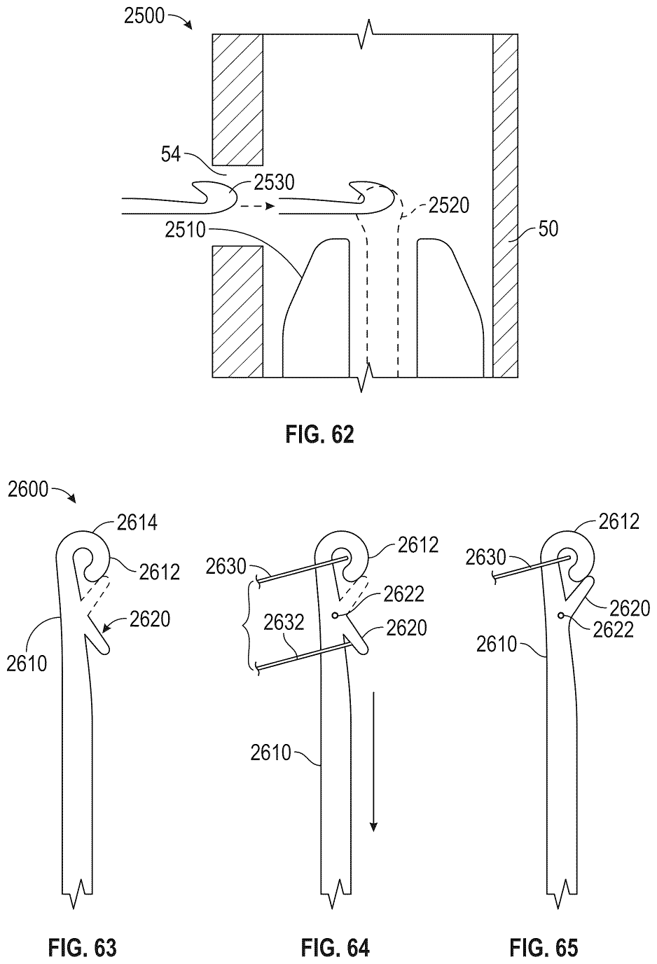

Referring now to FIG. 62, another exemplary embodiment of a nail assembly 2500 is shown. Assembly 2500 includes a cannulated nail 2510 with a looped suture 2520 passing through and extending from a top end 2512 of nail 2510. A passage 54 is formed in bone 50 sized to allow the insertion of a hook 2530 therein. Hook 2530 can be advanced into the cavity in bone 50 to capture suture 2520 and pull suture 2520 through passage 54, where suture 2520 can be secured against an exterior of bone 50 by a button, similar to either button 930, 1030 described above.

FIGS. 63-65 show still another alternative embodiment of a nail assembly 2600. Assembly 2600 includes a nail 2610 with a hook 2612 at a top end 2614 thereof. A latch 2620 is provided below hook 2612. Latch 2620 is attached to nail 2610 at a pivot 2622.

After nail 2610 is inserted into the bone to a desired location, a first K wire 2630 is inserted into the bone under hook 2612 and a second K wire 2632 is inserted into the bone under latch 2620.

Nail 2610 is pulled downward so that latch 2620 engages K wire 2632 and pivots from the position shown in FG. 64 to the position shown in FIG. 65, capturing a suture inserted along K wire 2630. The suture can be pulled tight to secure nail 2610 to the bone.

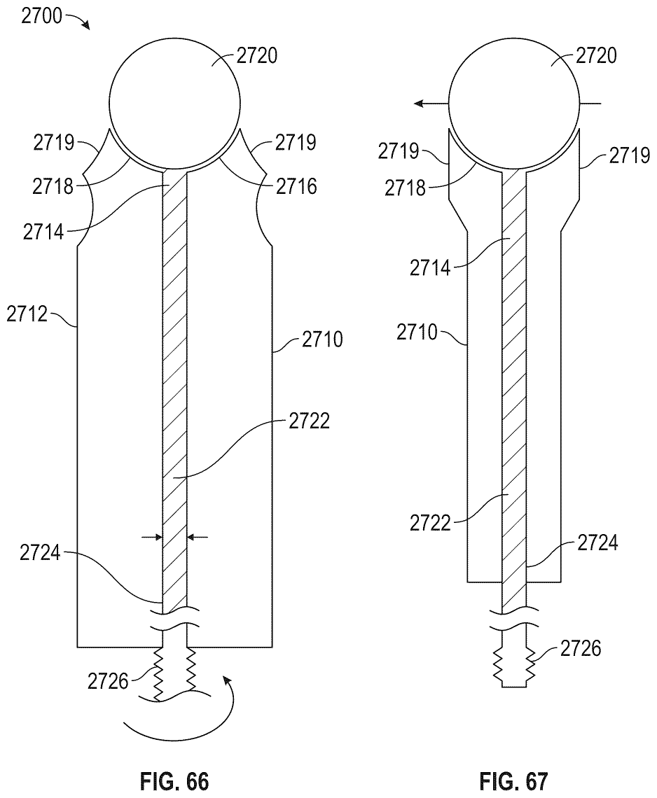

FIGS. 66-67 show still another alternative embodiment of a nail assembly 2700. Assembly 2700 includes a nail 2710 having a body 2712 with a through cannula 2714. A top end 2716 of body 2712 has a concave seat 2718 to seat a ball 2720. Top end 2716 below seat includes a plurality of barbs 2719.

A cable 2722 is attached to ball 2720 and extends through cannula 2714. Cable 2722 can be a pretensioned cable constructed from a shape memory material, such as Nitinol, with a bottom end 2724 attached to a set screw 2726 that is screwed into nail 2710. As cable 2722 warms up, cable 2722 shrinks, pulling ball 2720 downward and forcing barbs 2719 outward to dig into bone to prevent movement of nail 2710.

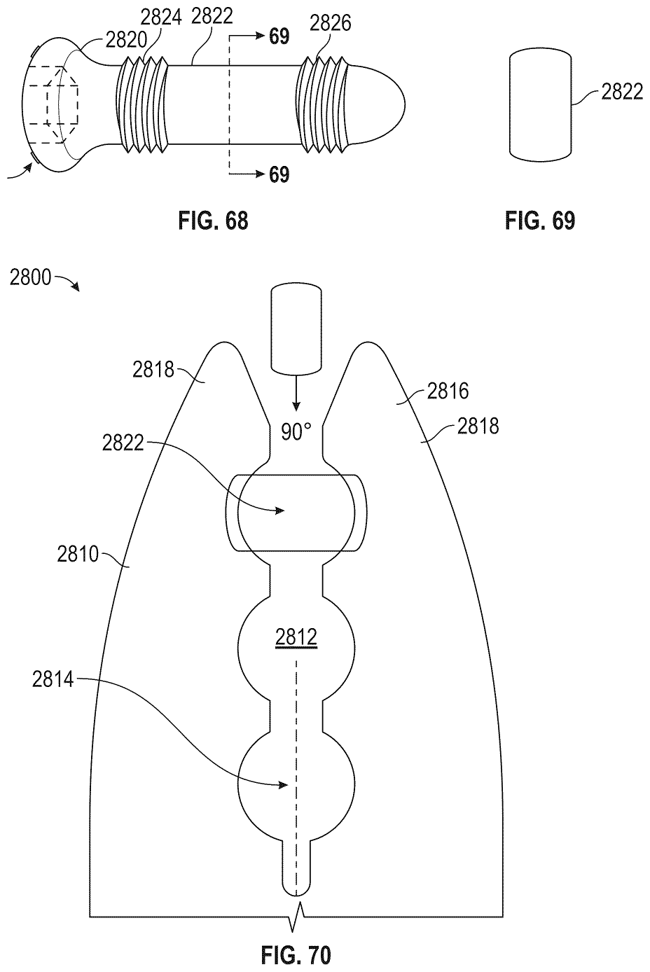

FIGS. 68-70 show yet another embodiment of a nail assembly 2800. Nail assembly 2800 includes a nail 2810 and screw 2820. Screw 2820 has a non-threaded portion 2822 between threads 2824, 2826. Non-threaded portion 2822 has a generally rectangular cross section, as seen in FIG. 69, such that the length is substantially wider than the width.

Nail 2810 includes a slot 2812 having an axis 2814 aligned to a handle or laser marking on distal end 2816 for aligning. Screw 2820 extends across slot 2812 such that, in a non-deployed position, the narrower width of portion 2822 allows side walls 2818 of nail 2810 to be biased toward each other so that nail 2810 can be advanced through the bone. When nail 2810 is in a desired location, screw 2820 is rotated 90 degrees so that non-threaded portion 2822 is forced between side walls 2818, biasing side walls 2818 against the inner wall of the bone, securing nail 2810 in place.

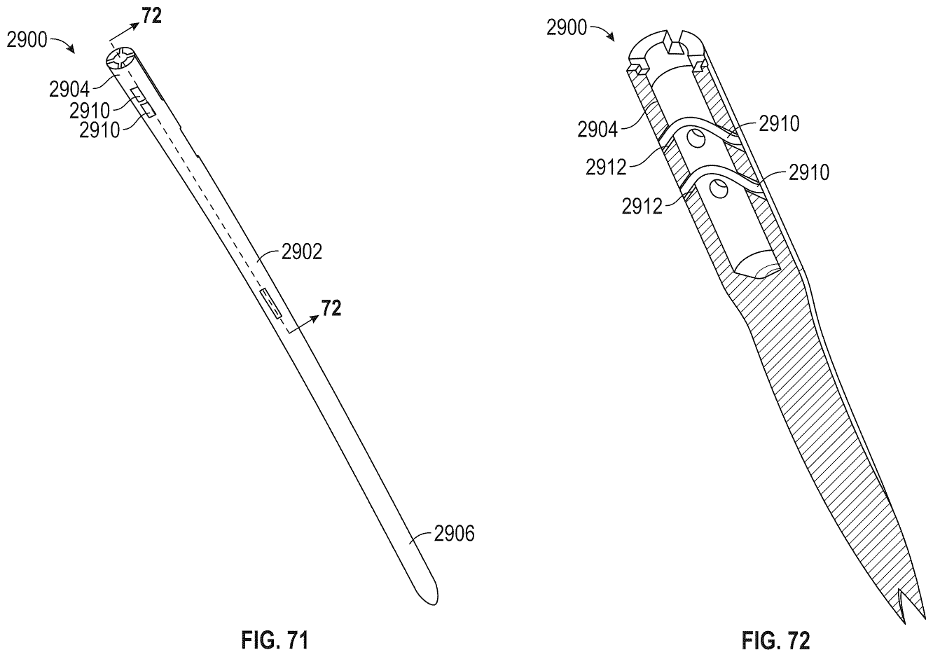

FIGS. 71-72 show still another embodiment of a nail assembly 2900. Nail assembly 2900 includes an elongate body 2902 having a proximal end 2904 and a distal end 2906. A pair of through openings 2910 are provided at proximal end 2904.

A cross in 2912 is inserted into each through opening 2910. Cross pins 2912 are constructed from a shape memory material, such as Nitinol. Cross pins 2912 are inserted into through openings 2910 in a straight condition, and are then bent prior to implantation by an insertion tool (not shown). When released, cross pins 2912 attempt to return to their original, straight condition, and pull bone segments together to generate compression between the bone segments.

It will be further understood that various changes in the details, materials, and arrangements of the parts which have been described and illustrated in order to explain the nature of this invention may be made by those skilled in the art without departing from the scope of the invention as expressed in the following claims.

We claim:

1. An intramedullary nail comprising:
a body having a proximal end and a distal body portion, and a longitudinal axis extending between the proximal end and the distal body portion; and
a means for locking the nail to a bone, the nail being inserted into the bone.

2. The intramedullary nail according to claim 1, wherein the locking means is in the proximal end.

3. The intramedullary nail according to claim 2, wherein the proximal end comprises at least one through passage extending through the longitudinal axis, the at least one through passage configured to receive the securing means.

4. The intramedullary nail according to claim 3, wherein the securing means comprises a shape memory material.

5. The intramedullary nail according to claim 3, wherein the at least one through passage extends perpendicularly to the longitudinal axis.

6. The intramedullary nail according to claim 1, wherein the means for locking the nail is in the proximal end.

7. The intramedullary nail according to claim 1, further comprising a cannula extending along the longitudinal axis.

8. The intramedullary nail according to claim 7, wherein the means for locking the nail is in the cannula.

9. The intramedullary nail according to claim 1, wherein the means for locking the nail comprises a cable.

10. The intramedullary nail according to claim 1, wherein the cable is configured to manipulate the proximal end.

11. The intramedullary nail according to claim 1, wherein the means for locking the nail comprises the proximal end being expandable away from the longitudinal axis.

12. The intramedullary nail according to claim 1, wherein the means for locking the nail is movable along the longitudinal axis.

13. The intramedullary nail according to claim 1, wherein the means for locking the nail is rotatable relative to the longitudinal axis.

14. The intramedullary nail according to claim 1, wherein the means for locking the nail pivots relative to the longitudinal axis.

15. The intramedullary nail according to claim 1, wherein the means for locking the nail comprises:
a locking mechanism in the proximal end; and
an activator for activating the locking mechanism in the distal end portion.

16. The intramedullary nail according to claim 1, wherein the means for locking the nail is integral with the nail.

17. The intramedullary nail according to claim 1, wherein the means for locking the nail is separate from the nail.

18. The intramedullary nail according to claim 1, wherein the means for locking the nail comprises a plurality of barbs extending from the proximal end.

19. The intramedullary nail according to claim 1, wherein the means for locking the nail is configured to be inserted into the bone with the body.

20. An intramedullary nail comprising:
a body having a proximal end and a distal body portion, and a longitudinal axis extending between the proximal end and the distal body portion; and
a means for locking the nail to a bone, the means extending between the proximal end and the distal end portion along the longitudinal axis.
